(19) United States
(12) Patent Application Publication
(10) Pub. No.: US 2007/0004681 A1
(43) Pub. Date: Jan. 4, 2007

(54) Methods for treating arthritic conditions

(76) Inventors: Le T. Duong, Lansdale, PA (US); Tadashi Hayami, Lansdale, PA (US); Anatasia G. Daifotis, Westfield, NJ (US); Gideon A. Rodan, Bryn Mawr, PA (US)

Correspondence Address: MERCK AND CO., INC, P O BOX 2000, RAHWAY, NJ, 07065-0907, US

(21) Appl. No.: 10/550,532

(22) PCT Filed: Mar. 23, 2004
(86) PCT No.: PCT/US04/08813

§ 371 Date: Sep. 22, 2005

Related U.S. Application Data
(60) Provisional application No. 60/457,974, filed on Mar. 27, 2003.

(51) Int. Cl. A61K 31/675; A61K 31/663

(52) U.S. Cl. 514/89; 514/102

(57) ABSTRACT

The present invention relates to a method for eliciting a disease modifying effect on an arthritic condition in a mammal which comprises administering to the mammal a therapeutically effective amount of an anti-resorptive compound. The present invention also relates to method for eliciting a disease modifying effect on subchondral bone sclerosis, preventing osteophyte formation or progression and preventing joint destruction in a mammal which comprises administering to the mammal a therapeutically effective amount of an anti-resorptive compound.

### BACKGROUND OF THE INVENTION

Osteoarthritis (OA) is a degenerative joint disease characterized by pain, cartilage loss and joint stiffness. Although OA has long been considered to be primarily a cartilage disorder associated with focal articular cartilage degradation, this disease is accompanied by a well-defined series of changes in the subchondral bone, including sclerosis, cyst formation and osteophyte formation, see, e.g., Oettmeier, R., and K. Abendroth, 1989, “Osteoarthritis and bone: osteologic types of osteoarthritis of the hip,” Skeletal Radiol. 18:165-74. The relative importance of the bone changes in the initiation and progression of OA is currently debated. Increased subchondral bone stiffness has been suggested to reduce its ability to dissipate load and distribute forces generated within the joint, subjecting the overlaying articular cartilage to greater peak dynamic forces, and thus accelerating its damage over time, see, e.g., Radin, E. L., and R. M. Rose, 1986, “Role of subchondral bone in the initiation and progression of cartilage damage,” Clin Orthop: 3440. The integrity of the articular cartilage therefore depends on the mechanical properties of the underlying bone. Cartilage damage does not always lead to full-thickness cartilage loss, which occurs only upon repetitive loading over an already stiffened subchondral bone plate, see, e.g., Burr, D. B., and M. B. Schaffler, 1997, “The involvement of subchondral mineralized tissues in osteoarthrosis: quantitative microscopic evidence,” Microsc Res Tech. 37:343-57. Recent studies have demonstrated an acceleration of subchondral bone turnover accompanied by specific architectural changes in the subchondral trabecular bone of OA joints, see e.g., Burr, D. B, 1998, “The importance of subchondral bone in osteoarthrosis,” Curr Opin Rheumatol. 10:256-62 and Goker, B., D. R. Sumner, D. E. Hurwitz, and J. A. Block, 2000, “Bone mineral density varies as a function of the rate of joint space narrowing in the hip,” J Rheumatol. 27:735-8. Furthermore, epidemiologic studies clearly documented increased subchondral bone sclerosis with disease progression, see Lane, N. E., and M. C. Nevitt, 2002, “Osteoarthritis, bone mass, and fractures: how are they related?” Arthritis Rheum. 46:1-4. The above hypothesis thus proposed an important role for subchondral bone in the initiation and progression of the disease process, suggesting that early intervention that reduces bone sclerosis might retard the progressive loss of articular cartilage.

The potential role of subchondral bone in the initiation and progression of OA has been the subject of controversy. Radin et al (1986) suggested that primary OA may start with changes in bone rather than cartilage. Remodeling of the subchondral bone plate exposed to excessive non-physiological mechanical loads results in stiffer bone of inhomogeneous density, which is no longer an effective shock absorber. Furthermore, above the denser and less compliant bone the physiological deformation of cartilage is impaired, which can generate shear stresses that result in cartilage damage, see Radin, E. L., and R. M. Rose, 1986, “Role of subchondral bone in the initiation and progression of cartilage damage,” Clin Orthop: 3440. On the other hand, cartilage damage does not always lead to OA if the tissue is not continuously exposed to impulsive loading over the stiffened subchondral bone plate, see Burr, D. B., and M. B. Schaffler, 1997, “The involvement of subchondral mineralized tissues in osteoarthrosis: quantitative microscopic evidence,” Microsc Res Teci. 37:343-57 and Burr, D. B, 1998, “The importance of subchondral bone in osteoarthrosis,” Curr Opin Rheumatol. 10:256-62 and Goker, B., D. R. Sumner, D. E. Hurwitz, and J. A. Block, 2000, “Bone mineral density varies as a function of the rate of joint space narrowing in the hip,” J Rheumatol. 27:735-8.

Emerging evidence supports the notion that bone turnover increases in OA, see Ratcliffe, A., and M. J. Seibel, 1990, “Biochemical markers of osteoarthritis,” Curr Opin Rheumatol. 2:770-6. An increase in urinary pyridinoline crosslinks and serum alkaline phosphatase and in the subchondral uptake of the radiolabeled bone marker ^{99m}Tc-MDP all point in that direction, see, Seibel, M. J., A. Duncan, and S. P. Robins, 1989, “Urinary hydroxy-pyridinium crosslinks provide indices of cartilage and bone involvement in arthritic diseases,” J Rheumatol. 16:964-70; Mansell, J. P., J. F. Tarlton, and A. J. Bailey, 1997, “Biochemical evidence for altered subchondral bone collagen metabolism in osteoarthritis of the hip,” Br J Rheumatol. 36:16-9; Dieppe, P., J. Cushnaghan, P. Young, and J. Kirwan, 1993, “Prediction of the progression of joint space narrowing in osteoarthritis of the knee by bone scintigraphy,” Ann Rheum Dis. 52:557-63. Since it is difficult to obtain biopsies from early OA patients, animal models have provided valuable insights into the disease process, including subchondral changes.

One aspect of osteophyte development is controlled by local levels of active TGF-β. TGF-β is a multifunctional cytokine, involved in fundamental biological processes such as development, extracellular matrix synthesis, cell proliferation/differentiation, and tissue repair, see Grimaud, E., D. Heymann, and F. Redini, 2002, “Recent advances in TGF-beta effects on chondrocyte metabolism. Potential therapeutic roles of TGF-beta in cartilage disorders.” Cytokine Growth Factor Rev. 13:241-57. In bone, TGF-β was suggested to be a physiological regulator of osteoblast differentiation and act as a mediator of the coupling between bone formation and resorption, during bone remodeling, see Erlebacher, A., E. H. Filvaroff, J. Q. Ye, and R. Derynck, 1998, “Osteoblastic responses to TGF-beta during bone remodeling,” Mol Biol Cell. 9:1903-18. TGF-β has been reported to have a dual role in the pathophysiology of OA, protection against cartilage damage and induction of osteophyte formation, see Grimaud, E., D. Heymann, and F. Redini, 2002, “Recent advances in TGF-beta effects on chondrocyte metabolism. Potential therapeutic roles of TGF-beta in cartilage disorders.” Cytokine Growth Factor Rev. 13:241-57 and van den Berg, W. B., P. M. van der Kraan, A. Scharstuhl, and H. M. van Beuningen, 2001, “Growth factors and cartilage repair,” Clin Orthop: S244-50. Active TGF-β is significantly up-regulated in synovial fluid in OA patients, see Schlaak, J. F., I. Pfers, K. H. Meyer Zum Buschenfelde, and E. Marker-Hemmann, 1996, “Different cytokine profiles in the synovial fluid of patients with osteoarthritis, rheumatoid arthritis and seronegative spondylarthropathies,” Clin Exp Rheumatol. 14:155-62 and Cameron, M. L., F. H. Fu, H. H. Paessler, M. Schneider, and C. H. Evans, 1994, “Synovial fluid cytokine concentrations as possible prognostic indicators in the ACL-deficient knee” Knee Surg Sports Traumatol Arthrosc. 2:3844. Local administration of TGF-β in the knee joints induced osteophyte formation, see Bakker, A. C., F. A. van de Loo, H. M. van Beuningen, P. Sime, P. L. van Lent, P. M. van der Kraan, C. D. Richards, and W. B. van den Berg, 2001, “Overexpression of active TGF-beta-1 in the murine knee joint: evidence for synovial-layer-dependent chondro-osteophyte formation,” Osteoarthritis Cartilage. 9:128-36.

### SUMMARY OF THE INVENTION

The present invention relates to a method for eliciting a disease modifying effect on an arthritic condition in a mammal which comprises administering to the mammal a therapeutically effective amount of an anti-resorptive compound. The present invention also relates to method for eliciting a disease modifying effect on subchondral bone sclerosis, preventing osteophyte formation or progression and preventing joint destruction in a mammal which comprises administering to the mammal a therapeutically effective amount of an anti-resorptive compound.

### DETAILED DESCRIPTION OF THE INVENTION

The present invention relates to a method for eliciting a disease modifying effect on an arthritic condition in a mammal which comprises administering to the mammal a therapeutically effective amount of an anti-resorptive compound.

The present invention relates to a method for eliciting a disease modifying effect on subchondral bone sclerosis in a mammal which comprises administering to the mammal a therapeutically effective amount of an anti-resorptive compound.

The present invention relates to a method for preventing osteophyte formation or progression in a mammal which comprises administering to the mammal a therapeutically effective amount of an anti-resorptive compound.

The present invention relates to a method for preventing or treating joint deterioration in a mammal which comprises administering to the mammal a therapeutically effective amount of an anti-resorptive compound.

The present invention relates to a method for eliciting a disease modifying effect on an arthritic condition in a mammal by inhibiting vascular invasion into calcified cartilage which comprises administering to the mammal a therapeutically effective amount of an anti-resorptive compound.

The present invention relates to a method for eliciting a disease modifying effect on subchondral bone sclerosis in a mammal by inhibiting vascular invasion into calcified cartilage which comprises administering to the mammal a therapeutically effective amount of an anti-resorptive compound.

The present invention relates to a method for preventing osteophyte formation or progression in a mammal by inhibiting vascular invasion into calcified cartilage which comprises administering to the mammal a therapeutically effective amount of an anti-resorptive compound.

The present invention relates to a method for preventing or treating joint deterioration in a mammal by inhibiting vascular invasion into calcified cartilage which comprises administering to the mammal a therapeutically effective amount of an anti-resorptive compound.

The present invention relates to the use of an anti-resorptive compound for the preparation of a medicament useful in the treatment of an arthritic condition. The present invention also relates to the use of an anti-resorptive compound and an agent selected from androgen receptor modulator; an inhibitor of osteoclast proton ATPase; an inhibitor of HMG-CoA reductase; an osteoblast anabolic agent; calcitonin; Vitamin K₂ or a pharmaceutically acceptable salts and mixtures thereof, for the preparation of a medicament useful in the treatment of an arthritic condition.

In an embodiment of the invention, the arthritic condition is amyloidosis; ankylosing spodylitis; bacterial arthritis; basic calcium phosphate crystal deposition disease; Behcet's disease; bursitis and tendinitis; CPPD deposition disease; calcific tendonitis; carpal tunnel syndrome; Ehlers-Danlos syndrome; enteropathic arthritis; Felty's syndrome; fibromyalgia; gout; fungal arthritis; hemoglobinopathy; hemophilic arthropathy; hypertrophic osteoarthropathy; infectious arthritis; inflammatory bowel disease; juvenile arthritis; juvenile rheumatoid arthritis; lupus erythematosus; lyme disease; marfan syndrome; mixed connective tissue disease; multicentric reticulohistocytosis, myopathies; myositis; osteoarthritis; osteonecrosis; osteonecrosischondrodystrophy; polyarteritis; polymyalgia rheumatica; psoriatic arthritis; Raynaud's phenomenon; reflex sympathetic dystrophy syndrome; Reiter's syndrome; relapsing polychondritis; rheumatoid arthritis; rheumatic fever; sarcoidosis; septic arthritis; scleroderma; Sjogren's syndrome; spondyloepiphyseal dysplasia; systemic lupus erythematosus; and viral arthritis.

Non-limiting examples of anti-resorptive compounds include, but are not limited to, organic bisphosphonates, cathepsin K inhibitors, integrin antagonists, estrogens, estrogen analogues, selective estrogen receptor modulators and combinations thereof.

“Organic bisphosphonate” includes, but is not limited to, compounds of the chemical formula 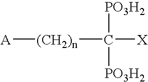
wherein n is an integer from 0 to 7 and wherein A and X are independently selected from the group consisting of H, OH, halogen, NH₂, SH, phenyl, C1-C30 alkyl, C3-C30 branched or cycloalkyl, bicyclic ring structure containing two or three N, C1-C30 substituted alkyl, C1-C10 alkyl substituted NH₂, C3-C10 branched or cycloalkyl substituted NH₂, C1-C10 dialkyl substituted NH₂, C1-C10 alkoxy, C1-C10 alkyl substituted thio, thiophenyl, halophenylthio, C1-C10 alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C3-C10 ring.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH₂, C1-C10 alkyl or dialkyl substituted NH₂, OH, SH, and C1-C10 alkoxy.

The foregoing chemical formula is also intended to encompass complex carbocyclic, aromatic and hetero atom structures for the A and/or X substituents, non-limiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C1-C30-alkyl-substituted ammonium Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, bisphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. For example, the phrase “about 5 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 5 mg of alendronic acid.

Non-limiting examples of bisphosphonates useful herein include the following:

Alendronate (also known as alendronic acid, 4-amino-1-hydroxybutylidene-1,1-bisphosphonic acid, alendronate sodium, alendronate monosodium trihydrate and 4-amino-1-hydroxybutylidene-1,1-bisphosphonic acid monosodium trihydrate) is described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

1-hydroxy-2-imidazo-1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate) is described in U.S. Pat. No. 5,583,122.

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zoledronate).

Non-limiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

It is recognized that mixtures of two or more of the bisphosphonate actives can be utilized.

Cathepsins belong to the papain superfamily of cysteine proteases. These proteases function in the normal physiological as well as pathological degradation of connective tissue. Cathepsins play a major role in intracellular protein degradation and turnover and remodeling. Cathepsin K (which is also known by the abbreviation cat K) is also known as cathepsin O and cathepsin O2. See PCT Application WO 96/13523, Khepri Pharmaceuticals, Inc., published May 9, 1996, which is hereby incorporated by reference in its entirety.

Cathepsin K is synthesized as a 37 kDa pre-pro enzyme, which is localized to the lysosomal compartment and where it is presumably autoactivated to the mature 27 kDa enzyme at low pH. See McQueney, M. S. et al., 1997, J Biol Chem 272:13955-13960; Littlewood-Evans, A. et al., 1997, Bone 20:81-86, which are hereby incorporated by reference in their entirety. Cathepsin K was cloned and found specifically expressed in osteoclasts See Tezuka, K. et al., 1994, J Biol Chem 269:1106-1109; Shi, G. P. et al., 1995, FEBS Lett 357:129-134; Bromme, D. and Okamoto, K., 1995, Biol Chem Hoppe Seyler 376:379-384; Bromme, D. et al., 1996, J Biol Chem 271:2126-2132; Drake, F. H. et al., 1996, J Biol Chem 271:12511-12516, which are hereby incorporated by reference in their entirety, and it appears that cathepsin K is involved in osteoclast mediated bone resorption.

Non-limiting examples of cathepsin K inhibitors can be found in PCT publications WO 00/55126 to Axys Pharmaceuticals; WO 01/49288 and WO 03/075836 to Merck Frosst Canada & Co. and Axys Pharmaceuticals; and WO 02/069901 to Merck Frosst Canada & Co. which are hereby incorporated by reference in their entirety.

As used above, “integrin receptor antagonists” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂≈₁, α₅β₁, α₆≢₁ and α₆β₄ integrins. H. N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact noncovalently and bind extracellular matrix ligands in a divalent cation-dependent manner. The most abundant integrin on osteoclasts is α_{c}β₃ (>10⁷/osteoclast), which appears to play a rate-limiting role in cytoskeletal organization important for cell migration and polarization. The αᵥβ₃ antagonizing effect is selected from inhibition of bone resorption, inhibition of restenosis, inhibition of macular degeneration, inhibition of arthritis, and inhibition of cancer and metastatic growth.

Non-limiting examples of integrin receptor antagonists, and methods for their preparation, are found in U.S. Pat. No. 5,925,655 (issued Jul. 20, 1999), U.S. Pat. No. 6,211,184 (issued Apr. 3, 2001), U.S. Pat. No. 5,919,792 (issued Jul. 6, 1999), U.S. Pat. No. 5,952,792 (issued Sep. 14, 1999), U.S. Pat. No. 6,017,925 (issued Jan. 25, 2000), U.S. Pat. No. 6,048,861 (issued Apr. 11, 2000), U.S. Pat. No. 6,232,308 (issued May 15, 2001), U.S. Pat. No. 6,358,970 (issued Mar. 19, 2002), U.S. Pat. No. 6,040,311 (issued Mar. 21, 2000), U.S. Pat. No. 6,066,648 (issued May 23, 2000), U.S. Pat. No. 6,211,191 (issued Apr. 3, 2001), U.S. Pat. No. 6,017,926 (issued Jan. 25, 2000), U.S. Pat. No. 6,090,944 (Jul. 18, 2000), U.S. Pat. No. 6,410,526 (issued Jun. 25, 2002), U.S. Pat. No. 6,413,955 (issued Jul. 2, 2002), U.S. Pat. No. 6,426,353 (issued Jul. 30, 2002), U.S. Pat. No. 6,444,680 (issued Sep. 3, 2002), and in PCT International Publication Numbers WO 00/48603 (published Aug. 24, 2000), WO 01/53297 (published Jul. 26, 2001), WO 01/53262 (published Jul. 26, 2001), WO 02/22616 (published Mar. 21, 2002), WO 02/07730 (published Jan. 31, 2002), WO 02/28840 (published Apr. 11, 2002), WO 02/40505 (published May 23, 2002).

“Selective estrogen receptor modulators,” or SERMs, refers to compounds which interfere or inhibit the binding of estrogen to the receptor, regardless of mechanism. SERMs can modulate estrogen receptor alpha, beta or both. Examples of estrogen receptor modulators include, but are not limited to, estrogen, progestogen, estradiol, droloxifene, raloxifene, lasofoxifene, basedoxifene (TSE-424), tamoxifen, idoxifene, arzoxifene (LY353381), LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2 dimethylpropanoate, 4,4′-dihydroxybenzophenone-2,4-dinitrophenyl-hydrazone, and SH646. Examples of estrogen receptor beta agonists can be found in International Publication Nos. WO 01/82923 (published Nov. 8, 2001), WO 02/41835 (published May 30, 2002), WO 02/091993 (published Nov. 21, 2002) and WO 03/015761 (published Feb. 27, 2003) assigned to Merck & Co., Inc. which are hereby incorporated by reference in their entirety.

Combinations of anti-resorptive compounds with other agents useful in treating or preventing the disorders disclosed herein are within the scope of the invention. A person of ordinary skill in the art would be able to discern which combinations of agents would be useful based on the particular characteristics of the drugs and the disease involved. Such agents include the following: an androgen receptor modulator; an inhibitor of osteoclast proton ATPase; an inhibitor of HMG-CoA reductase; an osteoblast anabolic agent, such as PTH; calcitonin; Vitamin K₂; and the pharmaceutically acceptable salts and mixtures thereof.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999), which is hereby incorporated by reference in its entirety.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR® see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL® see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL® see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

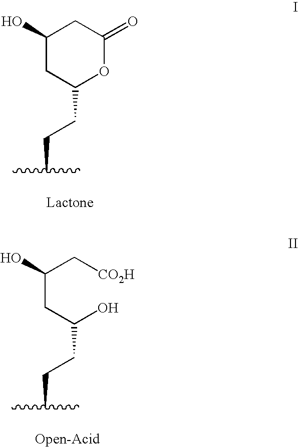

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically-acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl)aminomethane. Further examples of salt forms of HMG-CoA reductase inhibitors may include, but are not-limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

“An osteoblast anabolic agent” refers to agents that build bone, such as PTH. The intermittent administration of parathyroid hormone (PTH) or its amino-terminal fragments and analogues have been shown to prevent, arrest, partially reverse bone loss and stimulate bone formation or progression in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by R M Neer et al., in New Eng J Med 344 1434-1441 (2001).

In addition, parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)] and may also have potential as anabolic agents for treating osteoporosis.

Calcitonin is a 32 amino acid pepetide produced primarily by the thyroid which is known to participate in calcium and phosphorus metabolism. Calcitonin suppresses resorption of bone by inhibiting the activity of osteoclasts. Thus, calcitonin can allow osteoblasts to work more effectively and build bone.

“Vitamin K₂,” one of the two K vitamins, is a mixture of homologous fatsoluble substituted naphthoquinones (menaquinones), (C₁₁H₇O₂[C₅H₈]ₙH), where n may be 1 to 13 but is mostly 7 to 9. The term is also used for synthetic compounds resembling vitamin K₂ and having the same physiological action. The individual components of vitamin K₂ are also referred to by the number of isoprenyl units in the side chain (the number n in the formula), as for menadione, having no units at that position of the naphthaquinone ring, and also called vitamin K₂₍₀₎ (and also called vitamin K₃). It has been suggested that vitamin K₂ treatment effectively prevents the occurrence of new fractures, see Shiraki M, et al., “Vitamin K₂ (menatetrenone) effectively prevents fractures and sustains lumbar bone mineral density in osteoporosis,” J Bone Miner Res. 2000 March; 15(3):515-21.

Definitions

“Arthritic condition” or “arthritic conditions” refers to a disease wherein lesions, some of which are inflammatory, are confined to the joints or any inflammatory conditions of the joints, most notably rheumatoid arthritis (Academic Press Dictionary of Science Technology; Academic Press; 1st edition, Jan. 15, 1992). An arthritic condition can be caused by inflammation, trauma or infection. The compositions of the present invention are also useful, alone or in combination, to treat or prevent arthritic conditions or symptoms/diseases involving arthritis, such as amyloidosis; ankylosing spodylitis; bacterial arthritis; basic calcium phosphate crystal deposition disease; Behcet's disease; bursitis and tendinitis; CPPD deposition disease; calcific tendonitis; carpal tunnel syndrome; Ehlers-Danlos syndrome; enteropathic arthritis; Felty's syndrome; fibromyalgia; gout; fungal arthritis; hemoglobinopathy; hemophilic arthropathy; hypertrophic osteoarthropathy; infectious arthritis; inflammatory bowel disease; juvenile arthritis; juvenile rheumatoid arthritis; lupus erythematosus; lyme disease; marfan syndrome; mixed connective tissue disease; multicentric reticulohistocytosis, myopathies; myositis; osteoarthritis; osteonecrosis; osteonecrosischondrodystrophy; polyarteritis; polymyalgia rheumatica; psoriatic arthritis; Raynaud's phenomenon; reflex sympathetic dystrophy syndrome; Reiter's syndrome; relapsing polychondritis; rheumatoid arthritis; rheumatic fever; sarcoidosis; septic arthritis; scleroderma; Sjogren's syndrome; spondyloepiphyseal dysplasia; systemic lupus erythematosus; and viral arthritis. Unlike rheumatoid arthritis, osteoarthritis is a connective tissue disease, with pathology arising from mechanical insult-induced articular cartilage degeneration, subchondral bone remodeling and limited synovitic inflammation response. The net outcome of these activities is joint deformity secondary to erosion of articular cartilage, peri-articular endochondral ossification/osteophytosis, subchondral bone sclerosis and cyst formation, see, Oettmeier, R., and K. Abendroth, 1989, “Osteoarthritis and bone: osteologic types of osteoarthritis of the hip,” Skeletal Radiol. 18:165-74; Cutolo M, Seriolo B, Villaggio B, Pizzorni C, Craviotto C, Sulli A. Ann. N.Y. Acad. Sci. 2002 June; 966:131-42; Cutolo, M. Rheum Dis Clin North Am 2000 November; 26(4):881-95; Bijlsma J W, Van den Brink H R. Am J Reprod Immunol 1992 October-December; 28(34):231-4; Jansson L, Holmdahl R.; Arthritis Rheum 2001 September; 44(9):2168-75; and Purdie D W. Br Med Bull 2000; 56(3):809-23. Also, see Merck Manual, 17th edition, pp. 449-451. An embodiment of the invention encompasses the treatment or prevention of an arthritic condition which comprises administering a therapeutically effective amount of a composition of the present invention. A sub-embodiment is the treatment or prevention of osteoarthritis which comprises administering a therapeutically effective amount of a composition of the present invention.

“Subchondral bone sclerosis” as used herein means the increase in bone density and volume in the subchondral region.

“Osteophyte” as used herein refers to newly formed bony structures located at the joint margins, and their occurrence is strongly associated with the late stage of OA progression. The current hypothesis is that osteophytes originate from activated periosteum leading to new cartilaginous outgrowths that eventually turns into bone by the process of endochondral bone formation.

“Joint destruction” as used herein refers to the destruction of articular cartilage.

“Vascular invasion” as used herein refers to newly formed blood vessels under pathological condition in tissues such as cartilage which does not have blood vessels under normal condition.

“Calcified cartilage” as used herein refers to mineralized layer of cartilage at the border between cartilage and bone.

The term “disease modifying effect” refers to an agent that can slow, retard or prevent the progression of a disease. For example, in the case of osteoarthritis, a disease modifying effect could include slowing the loss of cartilage and preventing osteophyte formation or progression.

The term “composition” as used herein is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

As used herein, the term “pharmaceutically acceptable salts” includes the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm Sci., 1977:66: 1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Utilities

The compositions and methods of the present invention are useful for eliciting a disease modifying effect on arthritic conditions, especially for eliciting a disease modifying effect on osteoarthritis and rheumatoid arthritis, including the prevention of subchondral bone resorption, osteophyte formation or progression and ultimately joint deterioration/destruction.

An embodiment of the invention is a method of treating or preventing the progression of osteoarthritis in a mammal in need thereof, comprising administering to the mammal a therapeutically effective amount of an anti-resorptive. It is known in the literature that osteoarthritis (OA) is accompanied with a well-defined changes in the joints, including erosion of the articular cartilage surface, peri-articular endochondral ossification/osteophytosis, and subchondral bony sclerosis and cyst formation, see Oettmeier R, Abendroth, K, “Osteoarthritis and bone: osteologic types of osteoarthritis of the hip:, Skeletal Radiol. 1989; 18: 165-74. Recently, the potential contribution of subchondral bone sclerosis to the initiation and progression of OA have been suggested. Stiffened subchondral bone as the joint responding to repetitive impulsive loading, is less able to attenuate and distribute forces through the joint, subjecting it to greater mechanical stress across the articular cartilage surface. This in turn accelerates cartilage wear and fibrillate, see Radin, E L and Rose R M, “Role of subchondral bone in the initiation and progression of cartilage damage”, Clin. Orthop. 1986; 213: 34-40. Inhibition of excessive subarticular bone resorption by an anti-resorptive agent could lead to inhibition of subchondral bone turnover, and thus may have a favorable impact on OA progression.

Another embodiment of the invention is a method of treating or preventing rheumatoid arthritic (RA) condition in a mammal in need thereof, comprising administering to the mammal a therapeutically effective amount of an anti-resorptive agent. It is known in the literature that progressive destruction of the periarticular bone is a major cause of joint dysfunction and disability in patients with rheumatoid arthritis (RA), see Goldring S R, “Pathogenesis of bone erosions in rheumatoid arthritis”. Curr. Opin. Rheumatol. 2002; 14: 406-10. In addition, generalized bone loss is a major cause of morbility associated with severe RA. The frequency of hip and spinal fractures is substantially increased in patients with chronic RA, see Gould A, Sambrook, P, Devlin J et al, “Osteoclastic activation is the principal mechanism leading to secondary osteoporosis in rheumatoid arthritis”. J. Rheumatol. 1998; 25: 1282-9. The use of anti-resorptive agents in the treatment or prevention of resorption in subarticular bone and of generalized bone loss represents a rational approach for pharmacological intervention on the progression of rheumatoid arthritis.

The methods of the present invention have an unexpected disease modifying effect in the treatment of arthritic conditions.

The compositions of the present invention can be administered in such oral dosage forms as tablets, capsules (each of which includes sustained release or timed release formulations), pills, powders, granules, elixirs, tinctures, sterile solutions or suspensions, syrups and emulsions. Likewise, it may also be administered in intravenous (bolus or infusion), intraperitoneal, topical (e.g., ocular eyedrop), intranasal, inhaled, subcutaneous, intramuscular or transdermal (e.g., patch) form, metered aerosol or liquid sprays, drops, ampoules, auto-injector devices or suppositories all using forms well known to those of ordinary skill in the pharmaceutical arts. An effective but non-toxic amount of the compositions desired can be employed. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen utilizing the compositions of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Advantageously, the compounds of the present invention may be administered in a single quarterly, monthly, weekly or daily dose, or the total daily dosage may be administered in divided doses of two, three or four times daily. Furthermore, the compound of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

The dosage of the anti-resorptive agent to treat the conditions disclosed in the present specification may be higher than the dose used to treat osteoporosis.

The precise dosage of the bisphosphonate will vary with the dosing schedule, the oral potency of the particular bisphosphonate chosen, the age, size, sex and condition of the mammal or human, the nature and severity of the disorder to be treated, and other relevant medical and physical factors. For humans, an effective oral dose of bisphosphonate is typically from about 1.5 to about 6000 μg/kg body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 40 mg of the alendronate compound, on an alendronic acid active weight basis, i.e. on the basis of the corresponding acid.

In alternative dosing regimens, the bisphosphonate can be administered at intervals other than daily, for example once-weekly dosing, twice-weekly dosing, biweekly dosing, twice-monthly dosing, bimonthly dosing, quarterly dosing (once every three months), semi-annually dosing (twice a year) and yearly dosing. In a once weekly dosing regimen, alendronate monosodium trihydrate would be administered at dosages of about 70 mg/week to about 280 mg/week. Nonlimiting examples of doses include 140 mg/week and 280 mg/week. The bisphosphonates may also be administered monthly, every six months, yearly or even less frequently, see WO 01/97788 (published Dec. 27, 2001), WO 01/89494 (published Nov. 29, 2001) and WO 03/095029 (published Nov. 20, 2003).

Oral dosages of the cathepsin K inhibitors of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion.

Oral dosages of the αvβ3 inhibitors of present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 10 mg to about 400 mg of active ingredient. Intravenously, the most preferred doses will range from about 0.1 to about 10 mg/kg/minute during a constant rate infusion.

According to a further aspect of the present invention, it may be desirable to treat any of the aforementioned conditions with a combination of an anti-resorptive agent and one or more other pharmacologically active agents suitable for the treatment of the specific condition. The anti-resorptive agent and the other pharmacologically active agent(s) may be administered to a patient simultaneously, sequentially or in combination. For example, the present compound may be employed directly in combination with the other active agent(s), or it may be administered prior, concurrent or subsequent to the administration of the other active agent(s). In general, the currently available dosage forms of the known therapeutic agents for use in such combinations will be suitable.

The compositions and methods of the present invention are administered and carried out until the desired therapeutic effect is achieved.

The identification of an anti-resorptive compound which is able to have utility in the present invention may be readily determined without undue experimentation by methodology well known in the art, such as the assay described herein.

### Assay

Materials and Methods

Osteoarthritis model and treatment—All procedures were carried out according to the Institutional Animal Care and Use Committee Guide in Merck Research Labs. Ninety-five 20-week old male Sprague-Dawley rats (Taconic, NJ) were used in the following experiments. Osteoarthritis (OA) model was surgically induced in 20-wk-old male rat knee joints or in 7-10 month old male NZ White rabbits. Briefly, the animals were anesthetized by isoflurane. The right knee joint was shaved, disinfected with iodine, and exposed though the medial parapatellar approach. The patella was dislocated laterally and the knee placed in full flexion. All operation procedures were performed using a surgical loupe. Anterior cruciate ligament (ACL) was transected with micro-scissors. To confirm complete transection of ACL, Lachman test was performed. After surgery, the joint surface was washed with sterile saline solution, and both capsule and skin were sutured using Vicryl 4-0 (Ethicon, Edinburgh, UK), absorbable suture and monofilament 4-0 Nylon threads (Ethicon, Edinburgh, UK). In Sham operation, the wound was closed by layers after subluxation of patella and saline washing. Buprenorphine hydrochloride (0.1 mg/kg) (Reckitt & Colman Products Ltd., Hull, England) was given as an analgesic. Animals were allowed to move freely in the soft bedding plastic cages.

A test compound was administered by either subcutaneous injection or orally dosing. Drug was dosed prior to the surgery in the prevention mode. In treatment mode, drug was dosed 1 or 2 weeks post-surgery. Endpoints were histological analysis, histomorphometry and evaluation of serum markers. In all studies, the animals were always included the following groups: ACL transection with vehicle, ACLT with a low and a higher doses of the drug, sham operation with vehicle, and sham operation with the high dose of the drug. Animals were sacrificed on 2- and 10-wk post-surgery with CO₂. In both time points, rats were injected 10-mg/kg calcein 3 days before the necropsy. In a separate study, the same groups of animals received either sham- or ACLT-operation and with or without drug treatment were used for TGF-β assay. These animals were sacrificed on 2-wk post-surgery.

Gross morphology, Tissue preparation and histology—After the disarticulation of the right joint, both femur and tibia were carefully cleaned free of muscles, and fixed in 4% paraformaldehyde (Fisher scientific, NJ) in phosphate buffer saline (PBS) for 24 hrs. Gross appearance of the distal femur was taken by digital camera (DIX, Nikon, Japan) with 1:4 Nikkor lens (Nikon, Japan) to evaluate osteophyte formation. Tibia was then cut in a half at the center of articular surface along with medial collateral ligament in frontal section with band saw (EXAKT technologies, Inc, Norderstedt, Germany). Anterior parts were re-immersed in 4% paraformaldehyde for another 24 hrs for paraffin embedding. Posterior parts were changed into 70% ethanol, and then embedded in methylmethacrylate. Sections at 5 μm thick were stained Masson's trichrome staining as described previously, see Gruber, H. E., G. J. Marshall, L. M. Nolasco, M. E. Kirchen, and D. L. Rimoin, 1988, “Alkaline and acid phosphatase demonstration in human bone and cartilage: effects of fixation interval and methacrylate embedments,” Stain Technol. 63:299-306 and Yamamoto, M., J. E. Fisher, M. Gentile, J. G. Seedor, C. T. Leu, S. B. Rodan, and G. A. Rodan, 1998, “The integrin ligand echistatin prevents bone loss in ovariectomized mice and rats” Endocrinology. 139:1411-9. Specimens were labeled with randomly assigned identification numbers to blind the investigator to the group designation during subsequent measurements.

For paraffin embedding, tissues were decalcified in 0.5 M ethylenedinitrilo-tetra acetic acid solution (pH 7.6, Fisher scientific, NJ) for 7 to 10 days, then treated with a graded ethanol series, followed by xylene, prior to embedding into paraffin wax (Fisher Scientific, NJ) as previously described, see Nakase, T., K. Takaoka, K. Hirakawa, S. Hirota, T. Takemura, H. Onoue, K. Takebayashi, Y. Kitamura, and S. Nomura, 1994, “Alterations in the expression of osteonectin, osteopontin and osteocalcin mRNAs during the development of skeletal tissues in vivo,” Bone Miner. 26:109-22 and Hayami, T., N. Endo, K. Tokunaga, H. Yamagiwa, H. Hatano, M. Uchida, and H. E. Takahashi, 2000, “Spatiotemporal change of rat collagenase (MMP-13) mRNA expression in the development of the rat femoral neck,” J Bone Miner Metab. 18:185-93.

Paraffin embedded specimen was sectioned and examined by histological analysis and immunohistochemistry. Paraffin sections were stained with toluidine blue-O (0.2% toluidine blue-O/0.1M sodium acetate buffer, pH 4.0) for proteoglycan content. Occasionally, sections were also stained with tartrate resistant acid phosphatase (TRAP) stain for osteoclast localization, as previously described, see Nakamura, Y., A. Yamaguchi, T. Ikeda, and S. Yoshiki, 1991, “Acid phosphatase activity is detected preferentially in the osteoclastic lineage by pre-treatment with cyanuric chloride,” J Histochem Cytochem. 39:1415-20.

Histopathological scores (modified Mankin score)—Semi-quantitative histopathological grading was performed according to a modified Mankin scoring system, which is a well established grading system in OA research, with some modifications, see Cake, M. A., R. A. Read, B. Guillou, and P. Ghosh, 2000, “Modification of articular cartilage and subchondral bone pathology in an ovine meniscectomy model of osteoarthritis by avocado and soya unsaponifiables (ASU),” Osteoarthritis Cartilage. 8:404-11; Little, C., S. Smith, P. Ghosh, and C. Bellenger, 1997, “Histomorphological and immunohistochemical evaluation of joint changes in a model of osteoarthritis induced by lateral meniscectomy in sheep,” J Rheumatol. 24:2199-209; Wenz, W., S. J. Breusch, J. Graf, and U. Stratmann, 2000, “Ultrastructural findings after intraarticular application of hyaluronan in a canine model of arthropathy,” J Orthop Res. 18:604-12.

Mankin score normally consists of five subcategories, including structure, chondrocyte number, chondrocyte clustering, proteoglycan content (stainability for toluidine blue-O), and subchondral plate and/or tidemark change including vascular invasion in cartilage. Since vascular invasion into cartilage was independently evaluated using Masson's trichrome staining, we omitted this category in the Mankin score. Three sections 100 μm apart were measured in each sample. Total possible score is 26 and scoring was done by a single observer with blinded according to a five-point scale (Cake et al. 2000). Low total score are consistent with minor degenerative cartilaginous lesions, whereas high total score indicative of more pronounced cartilaginous regions. In toluidine blue-O staining stainability, we use the terminology as previously described (Little, et al. 1997), “mild” was used when there was decreased toluidine blue-O staining with intact articular surface, “moderate” when there was decreased toluidine blue-O staining in association with surface fibrillation and clefts extending to but not below the middle zone, and “severe” when cartilage was lost down to the level of the calcified cartilage.

Bone histomorphometry—For quantification of the histological parameters, we used Image Pro plus (version 4, Media Cybernetics, MD) image analysis program. Images of articular cartilage and subchondral bone were examined using a Olympus fluorescence microscope (BX51, Japan) with ×4 objective lens and were recorded using a CCD/RGB color video camera (RT Slider SPOT, Diagnostic instrument. Inc., MI).

Histomorphometric measurements of both medial and lateral tibial plateaux were determined in two separate sections per knee joint, spaced 100 μm apart. Since subchondral region has been reported that affected in OA development, we developed a macro to measure subchondral bone volume per tissue area. Two areas from either medial or lateral tibial plateau, 600 μm depth×800 μm width, were measured with the center of the tibial plateau being semi-automatically determined according to the width of the tibial surface. To consistently place the area to be measured, the top of the rectangle always horizontally aligned along the surface of articular cartilage and its sides vertically aligned along the center line of the tibia. The data from two areas were combined for the medial or lateral tibial plateau, and measurements of 6 knees per group were averaged in each group.

Trabecular bone volume (BV/TV: percentage of endosteal bone and marrow compartment occupied by osteoid and mineralized bone) in subchondral region was measured by histomorphometric methods that complied with the nomenclature and were calculated according to the ASBMR guidelines, see Parfitt, A. M., M. K. Drezner, F. H. Glorieux, J. A. Kanis, H. Malluche, P. J. Meunier, S. M. Ott, and R. R. Recker, 1987, “Bone histomorphometry: standardization of nomenclature, symbols, and units,” Report of the ASBMR Histomorphometry Nomenclature Committee. J Bone Miner Res. 2:595-610. To detect active bone remodeling surfaces in the subchondral region, we also injected the rats with calcein (10 mg/kg) 3 days before necropsy. Labeled mineralized surfaces in the plastic sections can be viewed using the same Olympus fluorescence microscope as described above.

Vascular invasion into calcified cartilage—Vascular invasion into the calcified cartilage was quantified by counting the number of times the calcified cartilage contacted by subchondral marrow space as previously described, see O'Connor, K. M., 1997, “Unweighting accelerates tidemark advancement in articular cartilage at the knee joint of rats,” J Bone Miner Res. 12:580-9. The results from two sections, spaced 100 μm apart were measured.

Osteoclast score—TRAP positive cells were counted in calcified cartilage and osteophyte regions. The number of TRAP positive cells from two sections in each sample spaced 100 μm apart were measured and then averaged from 6 knees per group.

Osteophytes score and area—Osteophytes were defined as outgrowth of the bone and cartilage occurring at the joint margins in the tibial plateau. To evaluate incident of osteophyte formation (osteophyte score), total osteophyte number from 5 sections including 3 paraffin (anterior part of tibia) and 2 plastic sections (posterior part of tibia) at 100 μm apart, were evaluated from each knee joint. Surface area of each osteophyte was manually determined in Masson's trichrome stained sections using image pro analysis. Two sections, each section is 100 μm apart, were evaluated.

Serum and Urinary levels of COMP, CTX-I and CTX-II—Blood was obtained from cardiac puncture at each necropsy, 2- and 10-wk post-surgery. Serum samples were collected, and frozen in aliquots −70° C. Serum cartilage oligomeric matrix protein (COMP) were determined by AnaMar Medical AB (Uppsala, Sweden) using a modified enzyme-liked immunosorbent assay as previously described, see Larsson, E., A. Mussener, D. Heinegard, L. Klareskog, and T. Saxne, 1997, “Increased serum levels of cartilage oligomeric matrix protein and bone sialoprotein in rats with collagen arthritis,” Br J Rheumatol. 36:1258-61 and Saxne, T., and D. Heinegard, 1992, “Cartilage oligomeric matrix protein: a novel marker of cartilage turnover detectable in synovial fluid and blood,” Br J Rheumatol. 31:583-91. All determinations were done in duplicate.

Twenty-four-hour urine samples were collected from the individual animal's metabolic cages at 2 wk-post surgery. Samples were centrifuged and frozen in aliquots at −70° C. Assays for bone related degradation product from C-terminal telopeptide of type I collagen (CTX-I/Ratlaps, Nordic Bioscience Diagnostics, Denmark) were performed in our laboratory according to the manufacturer's instruction. Assays for cartilage related C-terminal telopeptide of type II collagen (CTX-II/CartiLaps) were performed by Nordic Bioscience Diagnostics, Denmark. Urinary creatinine determination was measured in each sample as a test for normal urinary output. CTX-I and CTX-II values were reported after normalized to the creatine concentration in the same sample.

Immunohistochemistry—Tissue sections were deparaffinized in xylene, hydrated in graded ethanol, then treated with 500 U/ml testicular hyaluronidase (Sigma, MO) at 37° C. for 20 min. Tissue sections were then incubated with using either anti-rat CD31 mAb (Endogen, MA), or anti-activated TGF-β, which recognizes only active form of TGF-β1, 2, and 3 (R &D) as described previously, see Fernandez, T., S. Amoroso, S. Sharpe, G. M. Jones, V. Bliskovski, A. Kovalchuk, L. M. Wakefield, S. J. Kim, M. Potter, and J. J. Letterio, 2002, “Disruption of transforming growth factor beta signaling by a novel ligand-dependent mechanism,” J Exp Med. 195:1247-55, anti-MMP-13 Ab, anti-MMP-9 Ab for over night at 4° C. as described previously, see Hayami, T., H. Funaki, K. Yaoeda, K. Mitui, H. Yamagiwa, K. Tokunaga, H. Hatano, J. Kondo, Y. Hiraki, T. Yamamoto, L. T. Duong, and N. Endo, 2003, “Expression of the cartilage-derived anti-angiogenic factor Chondromodulin-I decreases in the early stage of experimental osteoarthritis,” J. Rheumatol. (in press). In CD31 immunostaining, after rinsing in PBS with 0.3% Tween 20, they were incubated with biotin-conjugated anti-mouse Ab (LSAB2 kit, Dako, CA) for 10 min and followed with alkaline phosphatase-conjugated streptavidin for 10 min (Dako, CA). These sections were rinsed with PBS, and developed using fast red substrate system (Dako, CA) for 5 min and counterstained with hematoxyline. Double-labeled immuno-histochemical stainings with MMP-9/MMP-13 and TGF-β Abs were performed as previously described, see Hayami, T., H. Funaki, K. Yaoeda, K. Mitui, H. Yamagiwa, K. Tokunaga, H. Hatano, J. Kondo, Y. Hiraki, T. Yamamoto, L. T. Duong, and N. Endo, 2003, “Expression of the cartilage-derived anti-angiogenic factor Chondromodulin-I decreases in the early stage of experimental osteoarthritis,” J. Rheumatol. (in press). Briefly, tissue sections were incubated with TGF-β mAb, followed by AP-conjugated anti-mouse Ab, and developed to blue color with AP blue (Vector Laboratories, CA USA). They were washed twice with PBS with 0.3% Tween 20 for 1 hr, incubated with anti-MMP-9 or MMP-13 polyclonal Ab, followed by HRP-anti-rabbit Ab (DAKO, CA), and developed to brown color by 0.5 mg/ml 3,3′-diaminobenzidine tetrahydrochloride. As negative controls, the same procedures were carried out either without primary Ab or with mouse mAb IgG instead of primary antibody.

Mink Lung epithelial growth inhibition assay for TGF-β in supernatant from tibial plateaux/patellae organ culture—Patellae and tibial plateau were isolated from either ACLT—or sham operated joints with or without drug treatment. After disarticulation and dissection of the patellae, tibiae were carefully removed of soft tissue. Articular cartilage and subchondral bone tissue were cut by a bone saw (Buehler Isomet, IL) at 480 μm thickness from the articular surface. Dissected patellae and tibial plateaux were transferred to 24 well culture dishes, washed with 0.1% BSA α-MEM for 3 times, then incubated in same media at 37° C. under 5% CO₂. Supernatant after 12 hrs incubation was collected and frozen at −70° C. Active TGF-β was measured as described previously by using the mink lung epithelial cell bioassay, see Docagne, F., N. Colloch, V. Bougueret, M. Page, J. Paput, M. Tripier, P. Dutartre, E. T. MacKenzie, A. Buisson, S. Komesli, and D. Vivien, 2001, “A soluble transforming growth factor-beta (TGF-beta) type I receptor mimics TGF-beta responses,” J Biol Chem. 276:46243-50. Briefly, mink lung cells (Mv1Lu, ATCC, MD) were plated at 10,000 cells/well in 96-well CytoStar scintillating microplates (Amersham, NJ) in E-MEM, 10% FBS containing sodium pyruvate and non-essential amino acids. After 24 hrs, TGF-β1 was diluted in α-MEM (1:4) as final concentration and 50 μl was added to duplicate wells as a control, followed by adding condition media (50 μl/well). After 20 hrs, [¹⁴C-methyl]-thymidine was added to each well to a final dilution of 0.5 μCi/ml. Plates were counted after 4 hr and 24 hr. Data reported was from the 24 hr-time point.

Statistical analysis—Statistical comparisons were generated using Statview (SAS Institute Inc., NC). All data in tables 1-3 were shown as means±SD. Results are expressed as mean±SEM. Significance of difference between groups was evaluated with a one-way analysis of variance (ANOVA) to analyze variance across treatment groups, and Fisher's analysis of least significant difference (Fisher's PLSD) to compare treatment group means except where indicated. Difference in values was considered significant when p value was <0.05.

What is claimed is:

1. A method for eliciting a disease modifying effect on an arthritic condition in a mammal which comprises administering to the mammal a therapeutically effective amount of an anti-resorptive compound.

2. The method of claim 1 wherein the arthritic condition is osteoarthritis.

3. The method of claim 1 wherein the anti-resorptive compound is selected from a bisphosphonate, an integrin inhibitor, a cathepsin K inhibitor, a selective estrogen receptor modulator or a combination thereof.

4. The method of claim 3 wherein the bisphosphonate is alendronate or a pharmaceutically acceptable salt thereof.

5. The method of claim 4 wherein the alendronate is administered in a dosage of about 70 mg weekly to about 280 mg weekly.

6. The method of claim 5 wherein the alendronate is administered in a dosage of about 140 mg weekly.

7. A method for eliciting a disease modifying effect on subchondral bone sclerosis in a mammal which comprises administering to the mammal a therapeutically effective amount of an anti-resorptive compound.

8. The method of claim 7 wherein the anti-resorptive compound is selected from a bisphosphonate, an integrin inhibitor, a cathepsin K inhibitor, a selective estrogen receptor modulator or a combination thereof.

9. The method of claim 8 wherein the bisphosphonate is alendronate or a pharmaceutically acceptable salt thereof.

10. The method of claim 9 wherein the alendronate is administered in a dosage of about 70 mg weekly to about 280 mg weekly.

11. The method of claim 10 wherein the alendronate is administered in a dosage of about 140 mg weekly.

12. A method for preventing osteophyte formation or progression in a mammal which comprises administering to the mammal a therapeutically effective amount of an anti-resorptive compound.

13. The method of claim 12 wherein the anti-resorptive compound is selected from a bisphosphonate, an integrin inhibitor, a cathepsin K inhibitor, a selective estrogen receptor modulator or a combination thereof.

14. The method of claim 12 wherein the bisphosphonate is alendronate or a pharmaceutically acceptable salt thereof.

15. The method of claim 14 wherein the alendronate is administered in a dosage of about 70 mg weekly to about 280 mg weekly.

16. The method of claim 15 wherein the alendronate is administered in a dosage of about 140 mg weekly.

17. A method for preventing joint deterioration in a mammal which comprises administering to the mammal a therapeutically effective amount of an anti-resorptive compound.

18. The method of claim 17 wherein the anti-resorptive compound is selected from a bisphosphonate, an integrin inhibitor, a cathepsin K inhibitor, a selective estrogen receptor modulator or a combination thereof.

19. The method of claim 18 wherein the bisphosphonate is alendronate or a pharmaceutically acceptable salt thereof.

20. The method of claim 19 wherein the alendronate is administered in a dosage of about 70 mg weekly to about 280 mg weekly.

21. The method of claim 20 wherein the alendronate is administered in a dosage of about 140 mg weekly.

22. A method for inhibiting vascular invasion into calcified cartilage in a mammal which comprises administering to the mammal a therapeutically effective amount of an anti-resorptive compound.

23. The method of claim 22 wherein the anti-resorptive compound is selected from a bisphosphonate, an integrin inhibitor, a cathepsin K inhibitor, a selective estrogen receptor modulator or a combination thereof.

24. The method of claim 23 wherein the bisphosphonate is alendronate or a pharmaceutically acceptable salt thereof.

25. The method of claim 24 wherein the alendronate is administered in a dosage of about 70 mg weekly to about 280 mg weekly.

26. The method of claim 25 wherein the alendronate is administered in a dosage of about 140 mg weekly.

27. A method for eliciting a disease modifying effect on an arthritic condition in a mammal by inhibiting vascular invasion into calcified cartilage which comprises administering to the mammal a therapeutically effective amount of an anti-resorptive compound.

28. The method of claim 27 wherein the arthritic condition is osteoarthritis.

29. The method of claim 28 wherein the anti-resorptive compound is selected from a bisphosphonate, an integrin inhibitor, a cathepsin K inhibitor, a selective estrogen receptor modulator or a combination thereof.

30. The method of claim 29 wherein the bisphosphonate is alendronate or a pharmaceutically acceptable salt thereof.

31. The method of claim 30 wherein the alendronate is administered in a dosage of about 70 mg weekly to about 280 mg weekly.

32. The method of claim 31 wherein the alendronate is administered in a dosage of about 140 mg weekly.

33. The method of claim 1 which further comprises an agent selected from an androgen receptor modulator; an inhibitor of osteoclast proton ATPase; an inhibitor of HMG-CoA reductase; an osteoblast anabolic agent; calcitonin; Vitamin K₂ or a pharmaceutically acceptable salts and mixtures thereof.
